(19) United States
(12) Patent Application Publication
(10) Pub. No.: US 2008/0300467 A1
(43) Pub. Date: Dec. 4, 2008

(54) SURGICAL WOUND RETRACTOR WITH REUSABLE RINGS

(76) Inventor: Robert W. Schaefer, Bolton, MA (US)

Correspondence Address: LAW OFFICES OF PAUL E. KUDIRKA, 40 BROAD STREET, SUITE 300, BOSTON, MA, 02109, US

(21) Appl. No.: 11/754,510

(22) Filed: May 29, 2007

(51) Int. Cl. A61B 1/32

(52) U.S. Cl. 600/208

(57) ABSTRACT

A sleeve-type wound retractor has at least two pieces: a reusable ring and a single-use sleeve. The reusable ring is sterilized before surgery in a conventional fashion. Before surgery and in a sterile environment, such as the operating room, the ring is fasted to one end of the sleeve to form a wound retractor. The other end of the sleeve has another ring which may be permanently attached to the sleeve or which may also be attached to the sleeve in the sterile environment. The completed wound retractor, comprising the two rings and the sleeve, functions as the conventional incremental wound retractor by winding the sleeve around the outer ring during use.

### BACKGROUND

Wound edge retractors are well known, and are typically used during surgery to expand incisions so that body parts under the incision may be accessed by the surgeon. One well-known type of wound edge retractor has an inner ring spaced from an outer ring with a flexible sheet of material formed into a sleeve extending between the rings and connected at opposite ends to the rings. During surgery, the inner ring is squeezed into an oblong shape, inserted through the wound and allowed to expand against the inside edge of the wound. The outer ring overlaps the outside edge of the wound to cause the sleeve to contact the sides of the wound and expand the wound for surgery. The sides of the wound are also protected by the sleeve from contamination during surgery.

In some of these sleeve-type wound retractors, the length of the sleeve can be incrementally adjusted by rolling the sleeve onto the circumference of the outer ring, in turn, by rotating the outer ring about its central axis. Such an arrangement is illustrated in FIGS. 1-4 which show a retractor 30 that includes an inner ring 32 and an outer ring 34 spaced from inner ring 32. An elongate sleeve 36 having two opposed ends 38 and 40 extends between inner and outer rings 32 and 34 with one opposed end 38 secured to inner ring 32 and the other opposed end 40 secured to outer ring 34. For example, opposed ends 38 and 40 can secured to inside surface 48, or the outside surface 46, of sleeve 36 by wrapping the material around a ring and heat-sealing the material to itself.

Typically sleeve 36 is a flexible tubular sheet having a substantially uniform circumference between ends 38 and 40 and a length L that depends on the type of surgeries to be performed with retractor 30. It is preferable that the sleeve length L be long enough to accommodate incisions in tissue having walls of different thickness I_{WT}. The diameter Dₛ of sleeve 36 will also vary depending on the size of the incision used in the surgeries to be performed with retractor 30.

As shown in FIG. 2, inner ring 32 typically has a substantially round cross-sectional shape and a diameter Dₗ and is capable of being squeezed into a substantially oblong shape for insertion into an incision, whereupon it returns back to its original shape. Outer ring 34 has a size which is substantially similar to that of inner ring 32 and typically has a diameter Dₒ and a cross-section that makes it easy for a surgeon to roll the ring around its central axis X.

In one incremental example found in U.S. Pat. No. 5,524,644, the outer ring has an oblate cross-section with opposed flat chordal surfaces which help the surgeon to rotate the ring about its central axis. In other retractors, the outer ring is provided with a pre-loaded rotational torque to assist with rotation of the outer ring about its central axis in order to roll the sleeve about the outer ring thereby adjusting the sleeve length. This pre-loaded torque is provided by forming the outer ring in a “mobius” configuration in which the circumference of the outer ring is rotationally offset or twisted by a predetermined amount about the central axis of the outer ring along the length of the ring. An example of this latter configuration is disclosed in U.S. Pat. No. 6,723,044.

FIG. 3 illustrates the retractor 30 after the inner ring 32 has been inserted into the wound and is in contact with the tissue inner surface 42. FIG. 4 shows the retractor 30 after the outer ring 34 has been rolled down into contact with the tissue outer surface 44. However, all of the above sleeve-type wound retractors are made for single use operation. Thus, the retractors are typically made of polymeric materials that are permanently fastened together. After the completion of a single surgical operation, a retractor is simply discarded. This arrangement provides for easy sterilization. However, such retractors are relatively expensive and, consequently, are not generally used in simple surgical operations and veterinary surgery.

### SUMMARY

In accordance with the principles of the invention, a sleeve-type wound retractor has at least two pieces: a reusable ring and a single-use sleeve. The reusable ring is sterilized before surgery in a conventional fashion. Before surgery and in a sterile environment, such as the operating room, the ring is fasted to one end of the sleeve to form a wound retractor. The other end of the sleeve has another ring which may be permanently attached to the sleeve or which may also be attached to the sleeve in the sterile environment. The completed wound retractor, comprising the two rings and the sleeve, functions as the conventional incremental wound retractor by winding the sleeve around the outer ring during use. After surgery is completed, the retractor is removed from the wound, the reusable ring is (or rings are) separated from the sleeve and the sleeve is discarded. The reusable rings are then sterilized in preparation for another use.

In one embodiment, each reusable ring is divided into two ring halves along a plane passing through the ring central axis. To assemble the wound retractor, the sleeve is passed through and folded over a first ring half. A second ring half is then fastened to the first ring half. Various mechanisms can be used to fasten the ring halves together. For example, small pegs in one ring half can penetrate the sleeve and snap into holes in the other ring half to hold the ring halves together.

In another embodiment, ring halves are held together by a plurality of metal clips. In still another embodiment, the ring halves are held together by a heat-shrinkable plastic band.

In yet another embodiment, the single use sleeve is folded back on itself and sealed to form a pocket at the sleeve end. The reusable portion of the retractor is a straight piece of ring material with a generally cylindrical shape with couplers affixed to each end. One end of the ring material is fed through a slot in the pocket and threaded around the end of the sleeve. When the end of the ring material emerges out of the slot after passing around the end of the sleeve, the two ends are then coupled together for use. After surgery is completed the ends of the ring material are uncoupled and the ring material is slid out of the pocket. The sleeve can then be discarded and the ring material sterilized for further use.

In further embodiments, the ends of the ring in the previous embodiment are coupled together by a ball-and-socket arrangement. Other coupling arrangements such as a threaded stud attached to one ring end and a threaded socket in the other ring end can also be used.

### BRIEF DESCRIPTION OF THE DRAWINGS

FIG. 1 shows the overall construction of a conventional incremental wound retractor comprised of two rings and a sleeve.

FIG. 2 is a cross-sectional diagram of the conventional retractor taken along section lines 2-2 in FIG. 1 illustrating the manner of attaching the rings to the sleeve.

FIG. 3 is a diagram illustrating the conventional sleeve inserted into a wound.

FIG. 4 is a diagram of the conventional sleeve in a wound “rolled down” to expand the wound edges.

FIG. 5A is a diagram of a reusable ring that is divided into two ring halves along a plane passing through the ring central axis. One ring half has a set of small pegs which fit into a corresponding set of small holes in the other ring half to hold the rings halves and the sleeve together.

FIG. 5B is a diagram of the underside of the upper ring half showing the set of holes.

FIG. 6 is a partial cross-section of the pair of ring halves showing a peg and hole in more detail.

FIG. 7 is a schematic perspective view of the ring halves with the sleeve installed.

FIG. 8 is a cross-sectional diagram of the ring and sleeve assembly after the ring halves have been snapped together illustrating how the ring halves holds the sleeve.

FIG. 9 is a cross-sectional diagram of the ring and sleeve assembly after the ring halves have been snapped together and the ring has been rolled down to shorten the sleeve.

FIG. 10 is a perspective view of another embodiment of the invention in which clips are used to hold the ring halves together.

FIG. 11 is a cross-sectional view of a two-piece ring held together with a clip.

FIG. 12 is a cross-sectional view of a two-piece ring held together with a heat-shrinkable plastic band.

FIG. 13 is a perspective view of another embodiment in which a ring is formed by a piece of ring material that is slid into a pocket formed at the end of the sleeve.

FIG. 14 is a perspective view of the embodiment of FIG. 12 with the ring material partially inserted into the pocket.

FIG. 15 is a perspective view of another embodiment in which the ring material ends of the embodiment shown in FIGS. 12 and 13 are joined by a ball-and-socket arrangement.

FIG. 16 is a perspective view of still another embodiment in which the ring material ends of the embodiment shown in FIGS. 12 and 13 are joined by a threaded stud and socket arrangement.

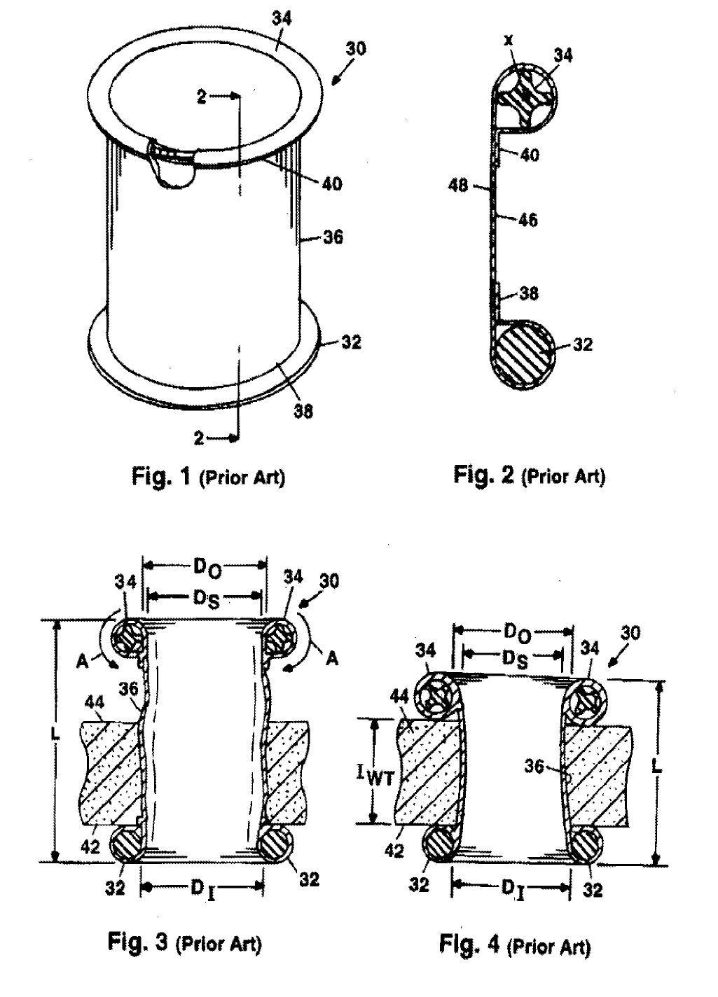

### DETAILED DESCRIPTION

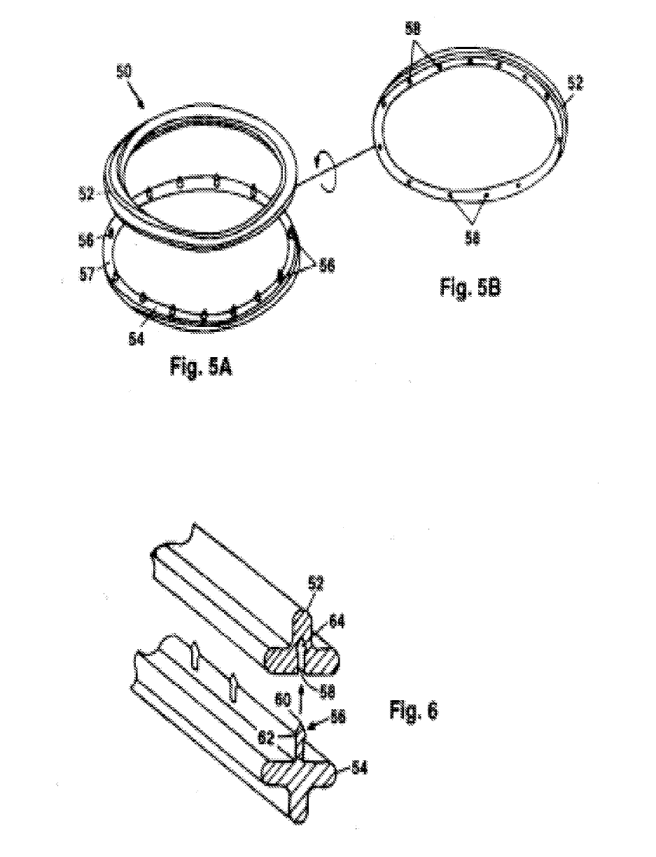

FIGS. 5A and 5B show one embodiment of the invention in which a reusable ring 50 is divided into two ring halves, 52 and 54, along a plane passing through the central axis of ring 50. Ring half 54 has a set of pegs 56 protruding from its upper surface 57. The pegs 56 may be evenly spaced around the circumference of ring half 54 or may be arranged in a predetermined pattern along the circumference. The set of pegs 56 fit into a set of holes 58 that are shown in more detail in FIG. 5B, which illustrates the underside of ring half 52. In accordance with the principles of the invention, the ring halves 52 and 54 may form a ring 50 that has various cross-sections. For example, the ring halves 52 and 54 may form a ring with a circular cross-section. Alternatively, as shown in he embodiment illustrated in FIGS. 5A and 5B, ring halves 52 and 54 may form a ring that has a “mobius” configuration or any other configuration that is easy for a surgeon to twist and roll in order to adjust the sleeve length as discussed above.

FIG. 6 shows a partial cross-section of the ring halves 52 and 54 showing the pegs and holes in more detail. As illustrated, each peg 56 has a pointed end 60 that facilitates its penetration through the sleeve material during the assembly of the rings and sleeve. Peg 56 also has an enlarged head 62 to cause it to snap into the corresponding recess 64 of hole 58 and, thus, securely fasten the ring halves 52 and 54 together. Because ring halves 52 and 54 are typically fabricated from an elastomeric material, peg 56 and be inserted into hole 58 without using a large force.

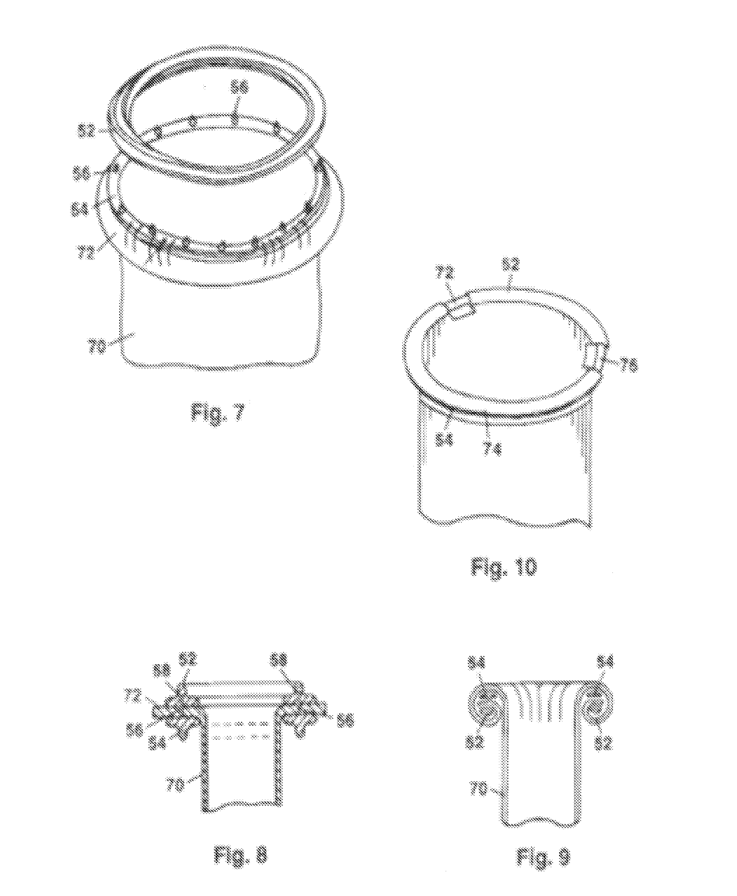

FIG. 7 shows the assembly of the wound retractor during which sleeve 70 is passed through ring half 54 and the top section 72 is folded over ring half 54. During this process, the pegs 56 pierce the sleeve material 70 to attach the sleeve material to the ring half 54. The second ring half 52 is then fastened to the first ring half 54 by snapping the pegs 56 into the holes 58 (not shown in FIG. 7.). The assembled ring and sleeve is shown in FIG. 8. FIG. 9 shows the assembly in a configuration where the ring is partially rolled down to reduce the sleeve length.

As in a conventional configuration, a second ring is provided at the other end of the single-use sleeve 70, which ring is not shown in FIGS. 5-9. The second ring can also be constructed as a removable ring using the construction shown in FIGS. 5-9 or may be permanently fastened to the sleeve as is the case with the conventional ring and sleeve design.

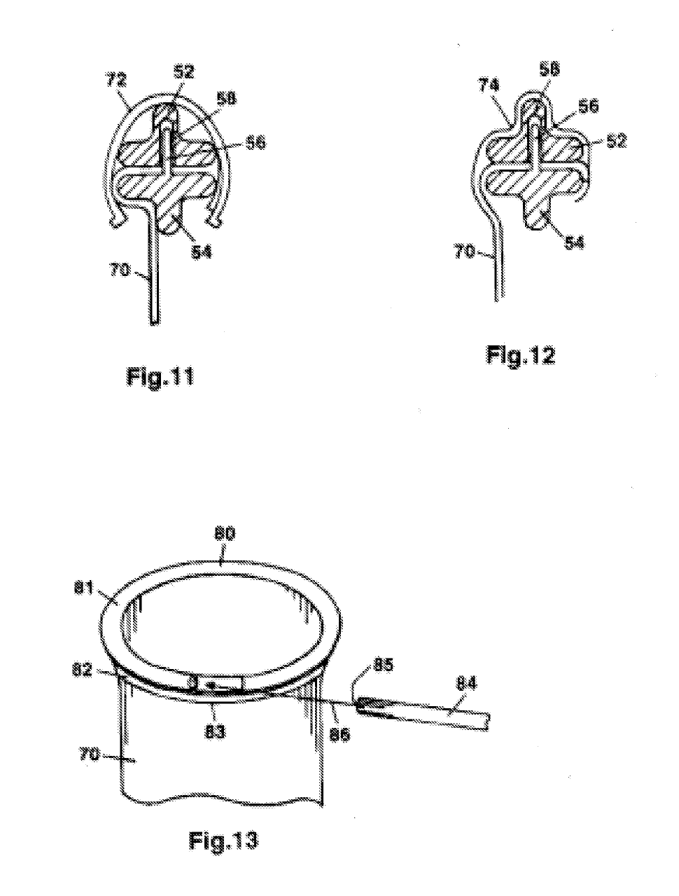

In another embodiment, ring halves 52 and 54 are held together by a plurality of metal clips rather than by snapping pegs 56 into holes 58. In this embodiment, pegs 56 and holes 58 are still present in order to fasten the sleeve 70 to the assembled ring, however, pegs 56 do not have enlarged heads which cause them to snap into holes 58. Instead, pegs 56 and holes 58 are cylindrical. This embodiment is illustrated in FIGS. 10 and 11. FIG. 10 is a perspective view of the assembled ring and sleeve assembly in which metal clips 72, 74 and 76 hold ring halves 52 and 54 together. Although three clips 72-76 are shown, those skilled in the art would understand that more, or fewer, clips could also be used. FIG. 11 is a cross-sectional view of the ring halves 52 and 54 as they are held together by clip 72. In still another embodiment shown in FIG. 12, the ring halves 52 and 54 are held together by a heat-shrinkable plastic band 74. Such a band can be activated after assembling the ring halves with a conventional hot air blower. Alternatively, the band can be activated by the heat of an autoclave used to sterilize the assembly. After use, the band is simply cut off and discarded.

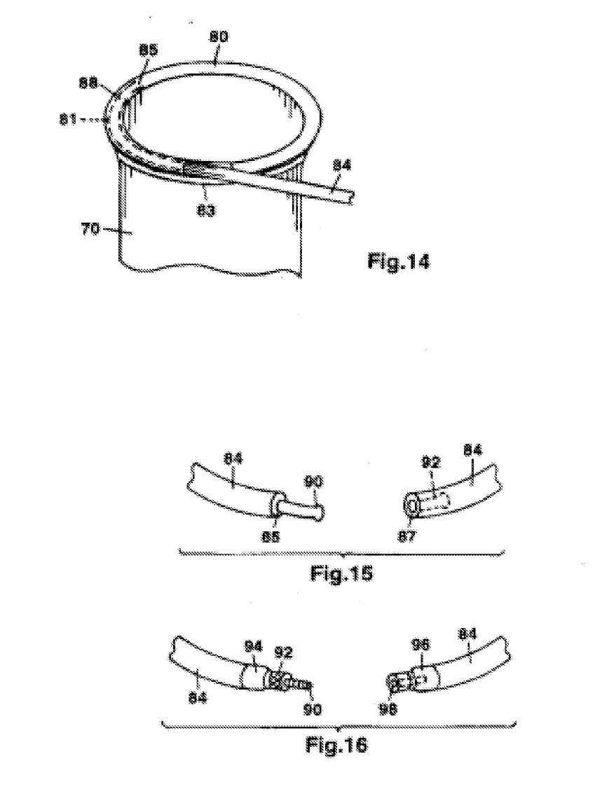

In yet another embodiment shown in FIGS. 13-16, the single use sleeve 70 is folded back on itself and sealed at 82 to form a pocket 80 at the sleeve end 81. The reusable portion of the retractor is a straight piece of ring material 84 with a generally cylindrical shape with couplers affixed to each end. One end 85 of the ring material 84 is fed through a slot 83 in the pocket 80 as indicated by arrow 86 in FIG. 13 and threaded around the end of the sleeve 70 as shown in FIG. 14. When the end 85 of the ring material 84 emerges out of the slot 83 after passing around the end of the sleeve 70, the two ends of the ring material 84 are then coupled together for use. After surgery is completed the ends of the ring material 84 are uncoupled and the ring material 84 is slid out of the pocket 80. The sleeve 70 can then be discarded and the ring material 84 sterilized for further use.

In a further embodiment, the ends of the ring material 84 in the previous embodiment are coupled together by a ball-and-socket arrangement as shown in FIG. 15. In this embodiment, the end 85 of material 84 is coupled to the end 87 of material 84 by means of a ball 90 which snaps into a socket 92. Another coupling arrangement is shown in FIG. 16 in which a threaded stud 90 which can be turned by a thumbwheel 92 is attached to one ring end by means of a metal cup 94. A threaded socket 98 in a metal cup 96 attached to the other ring end mates with the stud to hold the ring ends together.

While the invention has been shown and described with reference to a number of embodiments thereof, it will be recognized by those skilled in the art that various changes in form and detail may be made herein without departing from the spirit and scope of the invention as defined by the appended claims.

What is claimed is:

1. An adjustable surgical retractor, comprising:
an inner ring;
an outer ring spaced from the inner ring; and
an elongate sleeve open at opposite ends, the sleeve extending between the inner and outer rings and being connected at opposite ends to the rings wherein at least one of the inner ring and the outer ring is removably connected to the sleeve before use so that the one ring can be disconnected from the sleeve before discarding the sleeve and reused.

2. The surgical retractor of claim 1 wherein both the inner ring and the outer ring are removably connected to the sleeve before use and disconnected from the sleeve before discarding the sleeve.

3. The surgical retractor of claim 1 wherein the one ring comprises two ring halves each of which consists of a portion of the ring extending to one side of a plane intersecting the ring central axis.

4. The surgical retractor of claim 3 wherein the sleeve extends between the ring halves and the retractor further comprises a mechanism for fastening the ring halves together to removably connect the one ring to the sleeve.

5. The surgical retractor of claim 3 wherein the mechanism for fastening the ring halves together comprises a plurality of pegs formed on one ring half which fit into a corresponding plurality of holes in the other ring half.

6. The surgical retractor of claim 3 wherein the mechanism for fastening the ring halves together comprises at least one clip.

7. The surgical retractor of claim 3 wherein the mechanism for fastening the ring halves together comprises a heat-shrinkable plastic band.

8. The surgical retractor of claim 1 wherein the sleeve is folded back on itself and sealed to form a pocket at the sleeve end and the one ring comprises a straight piece of ring material that is fed through a slot in the pocket and threaded around the sleeve end.

9. The surgical retractor of claim 8 wherein the straight piece of material comprises couplers affixed to each end so that when an end of the ring material emerges out of the slot after passing around the sleeve end, the couplers are engaged to couple each end of the ring material together.

10. The surgical retractor of claim 9 wherein the couplers comprise ball-and-socket couplers.

11. The surgical retractor of claim 9 wherein the couplers comprise a threaded stud attached to one ring end and a threaded socket in the other ring end.

12. The surgical retractor of claim 1 wherein the outer ring is provided with a pre-loaded rotational torque to assist with rotation of the outer ring about its central axis to roll the sleeve about the outer ring to adjust sleeve length.

13. An adjustable surgical retractor, comprising:
an inner ring;
an outer ring spaced from the inner ring; and
an elongate sleeve open at opposite ends, the sleeve extending between the inner and outer rings and being connected at opposite ends to the rings, wherein one end of the sleeve is folded back on itself and sealed to form a pocket and the outer ring comprises a straight piece of ring material that is fed through a slot in the pocket and threaded around the one sleeve end so that the outer ring can be disconnected from the sleeve and reused and the sleeve discarded.

14. The surgical retractor of claim 13 wherein the inner ring is removably connected to the sleeve before use and disconnected from the sleeve before the sleeve is discarded.

15. The surgical retractor of claim 13 wherein the straight piece of material comprises couplers affixed to each end so that when an end of the ring material emerges out of the slot after passing around the sleeve end, the couplers are engaged to couple each end of the ring material together.

16. The surgical retractor of claim 15 wherein the couplers comprise ball-and-socket couplers.

17. The surgical retractor of claim 15 wherein the couplers comprise a threaded stud attached to one ring end and a threaded socket in the other ring end.

18. The surgical retractor of claim 13 wherein the other end of the sleeve is folded back on itself and sealed to form a second pocket and the inner ring comprises a straight piece of ring material that is fed through a slot in the second pocket and threaded around the other sleeve end so that the inner ring can be disconnected from the sleeve and reused.

19. An adjustable surgical retractor, comprising:
an inner ring;
an outer ring spaced from the inner ring; and
an elongate sleeve open at opposite ends, the sleeve extending between the inner and outer rings and being connected at opposite ends to the rings wherein the outer ring comprises two ring halves through which one end of the sleeve passes, each ring half consisting of a portion of the outer ring extending to one side of a plane intersecting the outer ring central axis.

20. The surgical retractor of claim 19 wherein the outer ring is provided with a pre-loaded rotational torque to assist with rotation of the outer ring about its central axis to roll the sleeve about the outer ring to adjust sleeve length.
